(19) United States
(12) Patent Application Publication
(10) Pub. No.: US 2020/0143912 A1
(43) Pub. Date: May 7, 2020

(54) COMPUTER-IMPLEMENTED METHOD AND SYSTEM FOR CONDUCTING ADAPTIVE CLINICAL TRIALS

(71) Applicant: Alicia Paladin, (US)

(72) Inventor: Michael Joseph Rosenberg, Port Orange, FL (US)

(21) Appl. No.: 16/701,130

(22) Filed: Dec. 2, 2019

Related U.S. Application Data
(63) Continuation of application No. 12/102,992, filed on Apr. 15, 2008.
(60) Provisional application No. 60/926,577, filed on Apr. 27, 2007.

(51) Int. Cl. G16H 10/20; G16H 10/60; G16H 70/40; G16H 50/70; G06Q 50/22; G06Q 10/08; G06Q 10/06; G06Q 30/02; G06Q 30/06

(52) U.S. Cl. CPC G16H 10/20; G16H 10/60; G16H 70/40; G16H 50/70; G06Q 30/0605; G06Q 10/087; G06Q 10/06393; G06Q 30/02; G06Q 50/22

(57) ABSTRACT

A computer-implemented method and system are described for conducting an adaptive clinical trial at a plurality of geographically remote sites. The invention includes (1) collecting performance metric data from remote clinical sites; (2) processing, tracking, and validating such data at a processing location; (3) electronically reporting the data to a pre-programmed computer module; (4) determining in real time, by use of the pre-programmed computer module, whether procedures or parameters utilized in conducting the clinical trial require modification; (5) providing instructions, based on such determining, to modify the procedures or parameters utilized in conducting the clinical trial; and (6) modifying the procedures or parameters utilized in conducting the clinical trial, based on the applicable instructions. The invention is of particular relevance to adaptive clinical trials, which demand the ability to quickly collect, process, and respond to various forms of data in order to adjust actions such as randomization schedules, interim analyses, treatment arm pruning, editing subpopulations, and other adaptive measures.

### BACKGROUND OF THE INVENTION

### Field of the Invention

The invention relates to methods and systems for collection of various types of data, for being able to rapidly analyze and respond to such data (as well as corresponding meta-data), and to provide real-time reporting. The invention finds application in conducting clinical trials in the medical field, as well as in other management systems, by providing a fully integrated ability to handle the many collection, analytic, and reporting functions. Since the ability to respond to changing circumstances is a central part of being able to successfully manage such a program, the invention enables an “adaptive” approach to both management and strategic aspects of conducting clinical trials.

The invention includes methods and systems for flexibly collecting various types of data (e.g., via interview, machine-read means, or any other manual or automated means), and transmitting these data to a central site where the data are processed, validated against certain rules, tracked, and summarized. The invention also provides the ability to analyze and summarize incoming data as meta-data (that is, data “about” the data, such as time to response, error rates, and other measures). In addition, administrative functions such as payment for milestone events or for submission of data or resupply of materials to remote sites, and other functions are included. In addition, an interactive component of the invention allows for instant modification of certain parameters of the study being managed, such as patient allocation to disparate treatment groups. The invention further provides the capability of electronically managing submitted data to ensure that discrepancies are identified, as well as an interactive component for communicating with clinical sites to resolve data discrepancies, and a flexible, computer network-based (e.g., Internet or intranet) system of reporting on data and performance indicators to sites, organizations managing the trial, and individuals involved with oversight.

### DESCRIPTION OF THE RELATED ART

Effective management systems must rely on a means of timely collection of data and performance measures with respect to same (e.g., meta-data), a means of analyzing and distributing reports to parties involved with oversight or management, in a manner that presents relevant information to a variety of functional roles that may be geographically diverse, and a means of responding to actionable information. Clinical evaluations of new pharmaceuticals in particular often involve numerous evaluations at different stages of development that often involve many sites spread throughout different countries and time zones. Such studies generally involve collection of clinical data, which may be subjective or objective, and which may be collected by observation, impressions (including from patients), or direct measurement, as well as related activities such as laboratory evaluations of blood and other specimens and other types of evaluations (such as x-rays, scans and the like). These data can be quite complex, with typical studies often involving hundreds of thousands of data points. The effective conduct of such studies requires that data be collected in a manner such that accuracy can be confirmed, and such that any discrepancy in the incoming data is promptly resolved, before analysis can proceed. To the extent that data and meta-data are delayed from being accessible in a meaningful form, the major objectives of a study, as well as measures of day-to-day management, are hindered.

A major shortcoming of current management systems is that these generally lack the ability to provide very timely, actionable information that enables adaptive management of studies. In this context, “adaptive” means that the course of such clinical investigations could be altered based on experience as a study progresses. An interesting approach (albeit one that falls far short of being truly “adaptive”) is disclosed in U.S. Pat. No. 5,991,731, which is directed to an internet-based system for communicating data from individual clinical sites to a central location for determination of patient eligibility for a study, randomization of patients into different “arms” of the study, and generation of initial drug prescriptions—all in “real time” while the patient is still in a physician's office. However, such a system in not really adaptive, because it allows only manual modification of one very small part of a study. It would be desirable if adaptive management could be applied much more broadly in the conduct of complex studies. For example, with knowledge of progress markers such as enrollment rates, successful strategies could be quickly identified and disseminated. Similarly, when performance of remote sites can be closely monitored, problems at one or more sites could be quickly identified and addressed, so as to minimize any negative consequences. Adaptive management also might be applied with respect to specific strategic elements of a study, such as discontinuing a dosing group when one “arm” of the study is demonstrated to be less efficacious and/or less safe than other treatment groups—a process known as “pruning.” Similarly, a Bayesian approach may be taken such as when the ratio of assignment to different treatment groups is altered depending on an outcome—a process known as “adaptive randomization.” In each case, adaptive management would require the very timely knowledge of data and performance measures.

Thus, there is a need for very rapidly collecting complex data from a variety of sources. The data itself can be used for two purposes: (1) to be able to effectively manage a process or processes, in order to optimize results, minimize errors, and produce a maximally efficient means of assessing both actions and the results of interventions taken to improve those actions, and (2) to ensure that no errors are present in the data that have been collected and, to the extent that errors, inconsistencies, values that represent outliers, or any other irregularities may exist, to be able to check such incoming data against a set of established rules that determine whether such data are of a maximal (or even acceptable) level of quality. This basic foundation forms the essence of many management systems that include areas as diverse as manufacturing, provision of services, and research. These desirably include collecting both data that can be used directly for assessment of a product or service (for example, acceptable levels of service as rated by users or buyers) and data that can be used indirectly to improve the management system itself.

Such data typically can be recorded and transmitted to a central location in a number of ways. The data first may be manually recorded on paper, and then may be either machine-read or entered manually via a keyboard. Alternatively, “paperless” data may be entered directly into a computer at a remote site, in either manual or automated fashion. Therefore, it would be desirable to have a system that facilitates the collection of data in a real-time or batch mode, from a variety of manual and/or automated sources.

In addition, many such applications involve a high degree of complexity that extends beyond the mere collection of data and measurement of performance metrics. For example, clinical trials also involve collection of data from laboratories, x-ray or imaging (such as CT or MRI scans), specialized laboratory evaluations, and specialized patient assessments (such as cardiac scans) that may involve measurements by different groups that may also be geographically diverse, and the results of which may need to be provided both to a central monitoring site and to the individual clinical site caring for the patient. In such cases, the assurance of consistency of measurement, provision of interim or final results back to individual sites, and other measurements may comprise part of the evaluations necessary to complete a study, Such studies also may involve assessments of safety, and need to take into account the possibility that an ill patient enrolled in a clinical study may seek additional medical attention in a facility that is not participating in the study and that may not have even rudimentary computer capabilities. These requirements are made even more demanding by the realization that many evaluations extend over several years and may involve multiple patient visits and assessments. All such data must, according to US and international regulations, be assured of accuracy.

Thus, it is desirable to have a means of providing feedback and coordination for each patient involved in an evaluation of a pharmaceutical product. Similarly, many such complex systems involve multiple inputs, so that a means of coordinating an overview of a situation, with the additional ability to “drill down” to individual records and even individual measurements, is critical. The inability to do so, and thus the inability to provide timely feedback to the individuals and sites collecting data when errors are made, results in a greater degree of inaccurate information, which undermines the ability of a study to demonstrate efficacy of a pharmaceutical product. In addition, each data discrepancy results in a query that is returned to the clinical site, a process that is estimated to cost approximately $100 US per query to resolve, since it requires going back to patient records. With the possibility of many thousands of such queries occurring during even a modest clinical study, the direct costs of data discrepancies can be considerable. Finally, the additional effort that the sites have to expend to assure good quality (“clean”) data results in an insidious cost when valuable time is required to deal with multiple data errors. Moreover, the delays in resolving outstanding discrepancies at the end of the study can take weeks to months, further delaying study analysis and progression of a development program.

Data are currently processed by comparing incoming data against as many as several hundred validation rules. These types of checks generally fall into three groups: (1) Range checks, which assure that a value for a data field falls within expected parameters and that the data are of the proper type (e.g., generally alpha or numeric); (2) Consistency, where an answer to one question may limit the responses to another question (for example, if subject is male, number of pregnancies should equal zero or Not Applicable); and (3) Trend information, where parameters may be specified for rate of change of certain variables (for example, a hemoglobin value may be consistent for the first four study visits but then drops precipitously, or height may be recorded as significantly lower than at previous visits). Being able to assess this information very quickly and to provide feedback to the sites that collected the data is very important to being able to assure that similar errors are not repeated.

Prior systems have utilized a web-based means of collecting data, transmitting this over computer networks or telephone lines, and putting data in a database. See, for example, U.S. Pat. No. 6,496,827, which discloses a system wherein data are input at a computer at the time of collection, for transmission over the internet to a central data storage site or database. The data are input in real time, via a graphical user interface that also provides means for rudimentary validation of the data. Further validation of the data occurs at the central site, via comparison with other data already in the database. However, systems such as those disclosed in U.S. Pat. No. 6,496,827 are limited by a lack of flexibility in how data are collected; require separate systems to perform data validation; and do not track study performance metrics.

A secondary form of data is meta-data, which can be defined as additional data that can be used to measure various performance criteria associated with the primary data. Such performance measures are generally not measured at present and reflect the complexity of clinical evaluations. However, it would be desirable to be able to track detailed performance measures that include (but are not limited to) benchmarks such as the number of queries generated by a clinical site, time to respond to queries, time to submit data following a patient visit, and other quality measures. These meta-data serve as a basis to identify areas needing improvement such as training of site personnel as well as enrollment strategy and many other performance measures, and to enable resources to be focused where they are most needed. Providing these data to sites, study managers, and others also provides an immediate performance feedback and serves as a stimulus to improve performance.

A further, currently unmet need in the industry is the enablement of performance-based payments, including incentives or disincentives, for groups that interact with a clinical trial management system. For example, clinical sites could be paid a certain proportion of their total payment when data are received, and the balance could be paid when queries associated with those particular data have been resolved. This would reward those clinical sites where data are rapidly collected and validated, while penalizing those sites where there may be problems with the collection and/or validation of data.

Finally, it would be desirable to be able to facilitate site payments in a clinical trial, based on different medical tests or procedures that may have been performed. For example, in some types of studies such as those evaluating treatment for Alzheimer's disease, MRI or similar scans may be performed at certain sites and not others.

### SUMMARY OF THE INVENTION

The above-identified shortcomings of the prior art are remedied by the present invention, which specifically enables a new class of clinical trials known as “adaptive,” because such trials utilize very timely information about clinical outcomes, in order to affect the way the trials are conducted, typically by continuously monitoring outcomes and continuously adjusting the way the trial is conducted. As non-limiting examples, such trials may be adjusted by altering the allocation ratio of patients in the study, or by early termination of certain dosing arms in dose-finding studies, or by sample size reassessment midway through a study. Another example might be to focus the time of supervisory personnel on those sites where recruitment is slowest.

In a first embodiment of the invention, a method and a corresponding system are provided for centrally managing data in an adaptive clinical trial or other adaptive process that is conducted at a plurality of geographically remote sites according to a set of procedures or parameters. The invention includes:
(a) collecting data from patients or participants in the course of conducting the clinical trial or other process at a remote site;
(b) electronically transmitting the data from the remote site to a processing location;
(c) checking the transmitted data at the processing location for validity, in automated fashion against one or more pre-determined rules,
(d) electronically reporting the data to an entity capable of determining whether the data require correction or whether procedures or parameters utilized in conducting the clinical trial or other process require modification; and
(e) providing instructions, based on the reported data, to
   (i) correct the data, or
   (ii) follow or modify the procedures or parameters utilized in conducting the clinical trial or other process.

In a second embodiment, the invention includes a versatile method and system for collecting data in a real-time or batch mode, from a plurality of sources.

In another embodiment, the invention provides a method and system for rapid validation of collected data, as well as for providing rapid feedback to the sites that collected such data. This embodiment of the invention includes:

(i) transmitting, through the web, a description of each discrepancy or query relating to the data, the validation that triggered the query, and means for responding to such query;

(ii) measuring the number of queries per clinical site or per each question in a questionnaire; or

(iii) measuring response time of a clinical site to one or more queries.

In yet a further embodiment of the invention, a method and a corresponding system are provided for conducting adaptive management of clinical trials, comprising collecting and analyzing meta-data.

In an additional embodiment, the invention includes a method and corresponding system for providing performance-based payments to particular clinical sites. This capability provides an incentive for the site to work quickly, enabling studies to be completed faster. The flexibility of the inventive method and system also enables disincentives such as reduction in payment if data or corrections are received after a certain time, for example more than a certain number of days after the site originally collected such data, or if the data reflect high error rates, or based on other measures.

The invention further provides a method and system for tracking the performance of different medical tests and procedures at different clinical sites. For example, the invention enables a site to be paid upon receipt of MRI (magnetic resonance imaging) or CT scan reporting data by a central location to which the site has submitted such data.

### BRIEF DESCRIPTION OF THE DRAWINGS

FIG. 1 is a block diagram depicting a preferred embodiment of a clinical trials management system according to the invention.

FIG. 2 is a block diagram depicting a site supply management system according to the invention.

FIG. 3 is a flow chart showing the operation of the site inventory tracking module included in the system of FIG. 2.

FIG. 4 is a flow chart showing the operation of a site payment system according to the invention.

FIG. 5 depicts an automated system for providing financial performance incentives to individual clinical sites.

FIG. 6A illustrates an example, according to the invention, of a means for tracking the status of a clinical study; specifically, various “status” categories currently applicable to patients who were screened for the study.

FIG. 6B illustrates an expansion of the data presented in FIG. 6A with respect to the specific status category “Screen Failure.”

FIG. 6C illustrates an expansion of the data presented in FIG. 6A with respect to the specific status category “Early Discontinuer.”

FIGS. 7A and 7B are bar graphs showing, respectively, patient screening and enrollment visits, by month.

FIG. 8 is a Microsoft Internet Explorer® page, customized according to the invention, depicting the tracking of patient “screen failure rate” at certain clinical sites over time.

FIG. 9 is a bar graph depicting monthly patient enrollment information for a particular clinical site.

FIG. 10 is a Microsoft Internet Explorer® page, customized according to the invention, depicting an overview of all queries within a query management system.

FIG. 11 is a Microsoft Internet Explorer® page, customized according to the invention, for viewing and responding to a particular query within a query management system.

FIG. 12 is a Microsoft Windows® dialog box, customized according to the invention, reflecting the extent to which certain categories of patient demographic information have been monitored at a particular clinical site.

FIG. 13 illustrates a report form according to the invention, showing the number of patients (“subjects”) at various clinical sites, as well as the relative amounts of verified and unverified information with respect thereto.

FIG. 14 illustrates an expansion of the data presented in FIG. 13 with respect to specific patients (“subjects”) at a particular clinical site.

FIG. 15 illustrates an example, according to the invention, of a means for tracking numbers of data queries generated and resolved, respectively.

FIG. 16 illustrates an example, according to the invention, of a means for tracking the timeliness of data submission with respect to patients (‘subjects”) whose enrollment in the clinical trial has ended.

### DETAILED DESCRIPTION OF THE INVENTION

All references cited in this application are incorporated by reference herein in their entireties.

Clinical trials are generally highly complex processes that involve collection of many thousands of data elements from multiple clinical sites, laboratory facilities, regulatory agencies, and often outside vendors such as companies supplying test drugs. Many of the foregoing may be in different countries, which present the challenges of different cultures, languages, time zones, and other differences that complicate the ability to effectively manage such diverse participants in a clinical evaluation. The quality of the data collected in such circumstances is of paramount importance, because accurate data are necessary to demonstrate the efficacy and safety of any pharmaceutical product being evaluated. Failure to optimize data quality slows study progress; requires a greater number of patients in order to demonstrate an effect; impairs the ability of a manager to change a study based on what already has occurred in the course of the study; and slows regulatory filings because of additional time required to assure “clean” data.

The present invention provides a comprehensive, fully integrated method and corresponding system that allows a high degree of flexibility in being able to collect different types of data, including performance indices that are automatically measured and tracked by the system; a flexible, interactive system of validating such data and tracking critical information for the management of each study (such as payments to sites that are based on procedures performed, patient evaluations, number of patients, and the like): and an internee-based system of rapidly reporting a variety of indices that reflect study progress (such as amount of data received) or other parameters (such as quality of data, anticipated completion of enrollment, allocation ratios, and the like). The system and method are particularly useful for large, geographically diverse studies involving many sources of data (e.g., clinical sites, laboratories, and other facilities), enabling the user to know precisely the status of any patient data and to be able to have indices that reflect site performance in critical areas such as patient enrollment, patient status (e.g., data transmitted, degree of completeness, overdue visits) and in closely monitoring and managing site performance through indices such as number and type of discrepancies, “age” of discrepancies, and the like.

The invention integrates and simplifies a number of key elements that heretofore either have not existed or have been separate entities, unable to communicate status with a centralized means of monitoring performance. Close monitoring of performance is necessary for any complex system that requires management, and the presence of clinical sites that are often spread throughout the world makes such a task difficult. The invention advantageously provides such a capability.

A major advantage of the invention is that it enables both study data and performance metrics to be monitored in near-real time, on a continuous basis. In contrast, previously existing systems have engendered delays and major unknown components during the course of a study.

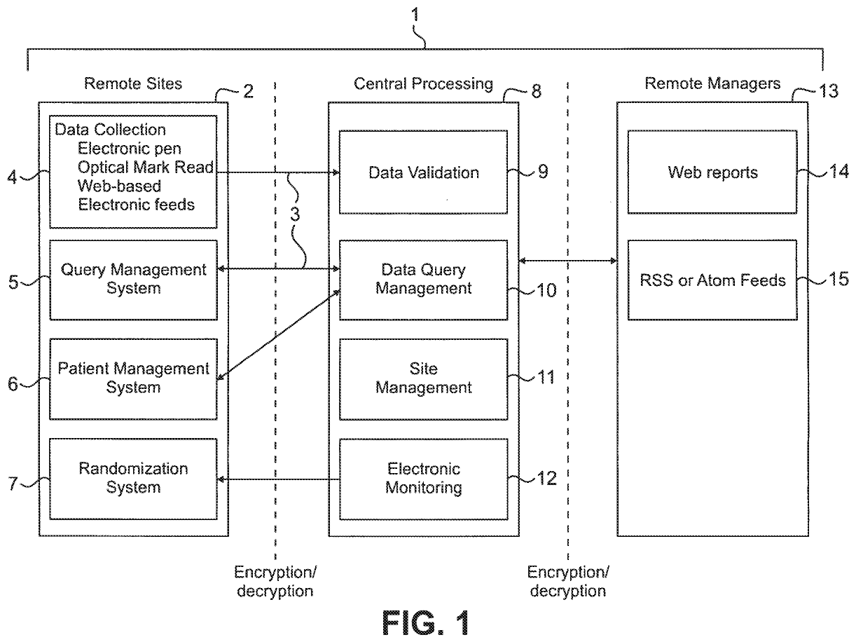

A preferred embodiment of a computerized system 1 for conducting a clinical study at a plurality of remote participating sites according to the invention is shown in FIG. 1, wherein each participating site has one or more host computers 2 for transmitting and receiving data over the Internet, as represented by uni-directional or bi-directional arrows 3. Each such host computer 2 includes data collection means 4 for receiving identification, demographic and medical data about the subjects from the respective participating site. Each data collection means 4 may comprise one or more data collection methodologies (human- or machine-collected), including optical mark reader forms, digital forms, web-based data entry and electronic feeds, as described further in the paragraphs below.

As shown in FIG. 1, system 1 further includes computer 8, located at a central processing site, which incorporates an algorithm or algorithms 9 for performing validation, or data consistency checks, for field, form and study-wide data errors. Queries relating to data validation or consistency are generated by computer 8 and are sent via data query management module 10 to the remote site, where responses are reviewed and rejected or approved utilizing query management system 5, an internet-based computer application that allows participating sites to respond to data queries. Data query management module 10 provides the ability to post queries that result from the data validation process to a secure website. This module 10 handles the display of queries and provides a mechanism whereby each query can be accessed and responded to on online. Additional capabilities for handling queries by this module include sorting by date or type, sorting according to which validation rule triggered the query, and other enhancements to facilitate handling and response by site and by monitors operating in the field.

Each host computer 2 in FIG. 1 further includes a patient management system 6 to generate reports on site activity, including patient visits, visit activity, screen failures (dates and reason), and patient discontinuation (dates and reason). In addition, each host computer 2 includes a randomization system 7, for randomizing drug dosages and for random assignment of participating patients into groups, including the handling of population stratification and double blind studies. These randomization events are tracked at the central processing site via electronic monitoring module 12.

Central processing site computer 8 further includes site management module 11, which enables study personnel to track enrollment rate, screen failures, queries, response times, and numerous other parameters of site performance. These parameters are valuable in determining the need for attention and its urgency, as well as for early detection of problems that may occur in sites. For example, site management module 11 provides central control over such activities as supply management and payments for each clinical site, as will be further described below.

Also as shown in FIG. 1, system 1 further includes a remote management capability including one or more computer systems 13 for study management staff to source-document-verify study data, to set up the clinical study applications, and to set up and maintain user access permissions to data repositories. Computer systems 13 also host a study web site 14 containing real-time reporting of clinical study data and data mining results reporting trends or biases. Similar status and results information is made available through web-feed formats 15, such as RSS or Atom, used to publish frequently updated digital content. These web-feed formats enable digital dashboards, “gadgets” and “widgets” that sit on a user's desktop.

Finally, as illustrated in FIG. 1, all data communicated between remote site computer systems 2, central processing site computers 8 and remote management computers 13 may be secured by encryption and decryption, utilizing methods standard in the art.

Critical components of the system of the invention (corresponding to system 1 in FIG. 1) are: (1) means of data collection; (2) a location for processing and database functions, which may be centralized; and (3) reporting functions. These components are described in detail in the following sections.

### Means of Data Collection and Transfer

Data may be collected in accordance with the invention by using any type of system that can produce an ASCII, CSV, or other type of file that associates a specific question to a specific response. These may include binary outcomes (yes/no or multiple choice), open-ended questions that record text or text strings, visual analogue scales (1-10 or similar), images (such as x-rays or CT scans), or any other means by which a specific response can be recorded (including, but not limited to, optical mark read, optical character recognition, electronic pen, tablet-based, web-based, and other data collection technologies).

In a preferred embodiment, the present invention utilizes an electronic pen for data entry, as illustrated in data collection means 4 in FIG. 1. The electronic pen (see, for example, U.S. Pat. Nos. 7,134,606 and 7,136,054) is a special pen that is equipped with an optical sensor that records pen strokes on a special gridded paper. There are many variations of the grid patterns, enabling the pen to determine which form it is on (through a master list that signifies which form is associated with each grid pattern) as well as the pen's orientation within that sheet of paper. This instrument then records each keystroke and, by specifying where the answer to each question is recorded, a question can be linked with a response. This information then can be transmitted over the internet, whereby software allows each stroke of the pen to be interpreted as numbers and/or letters which are then associated with a specific question and stored in a database.

Use of an electronic pen for data collection in the system and method of the present invention advantageously provides considerable savings of time and money. The main benefit of an electronic pen is that no keyboard entry is required; thus the data entry can be cheaper, faster, and more accurate. In practice, the electronic pen offers the capability of having data and corresponding meta-data completely entered and transmitted over the internet to a central location within minutes of a patient's visit to a clinical site—a major advantage over other systems that presently produce lag periods of between several days and several months.

Such other systems of data entry currently in use fall into two general categories: most (about 75% of current clinical studies) involve recording a value on a paper Case Report Form (“CRF”), after which it is entered by a data entry clerk or the like, who types each value into an electronic system, A second verification entry (“double key entry”) is then performed as a quality check. The other means of data entry, currently employed by approximately 25% of clinical trials, involves web-based Electronic Data Collection. This generally involves using a Worksheet onto which data are copied from Source Data, and from which the data then are keyed into an electronic system at the site. In either case, the need for manual data entry entails delays that are quite substantial as compared to the use of an electronic pen as contemplated in the present invention.

Use of an electronic pen for data collection in clinical trials, as taught herein, also would reduce the need for, and the expense associated with, field monitoring efforts. Field monitors (Clinical Research Associates, or “CRAB”) are an expensive resource for clinical trials. These are highly trained, well compensated individuals who normally visit field sites at regular and relatively short intervals because in the past, this was the only way to monitor progress, examine data, and manage remote sites. However, the need to utilize a good deal of time, including extensive travel time, of such individuals accounts for a considerable proportion of a trial's expenses.

Thus, in accordance with the invention, CRFs generated via electronic pen could be used as source documentation. Under the US Food and Drug Administration's Good Clinical Practices (“GCP”) that govern the conduct of all clinical trials, the first recording of a value (such as blood pressure reading, weight, or any other data collected as part of a clinical trial) is considered a “source document.” Typically, through a series of transcriptions and recordings, data eventually end up in a database that is used as the basis for analyzing study results. The final database values must be accurate, and industry practice is to have CRAs go to each clinical site and compare a database value against the source document where that value was first recorded—a process called Source Data Verification. However, because CRFs generated by an electronic pen could qualify as “source documentation,” and because the handling of such data would be entirely automated from that point forward and there is no possibility of transcription errors (and this electronic system is validated under 21 CFR Part 11—Guidance for Industry Computerized Systems Used in Clinical Trials), the use of an electronic pen would satisfy GCP requirements and therefore reduce or eliminate the need for Source Data Verification by highly paid experts.

In fact, by obviating the need for Source Data Verification, the use of an electronic pen can be expected to reduce the monitoring time requirements by approximately 80%, in addition to reducing the need for frequent site visits. Since field monitoring generally accounts for approximately one-third the cost of a clinical trial, the use of an electronic pen, in the context of the system and method of the present invention, could reduce the cost of a study by approximately 20%. Because the time required for transcription, data entry, and similar activities also would be reduced, considerable time savings also would result.

As mentioned above, optical mark read (“OMR”) is another data collection means suitable for use in the present invention. OMR is a method of rapidly scanning forms that have been completed by filling in “bubbles”; i.e., round response boxes. Users are familiar with this technology, since it has been long used on standardized tests that require very rapid and accurate interpretation and scoring for tests such as the Scholastic Aptitude Test. In OMR, special papers are used to allow scanners to orient themselves with regard to page margins and x-y coordinates within those margins. Responses are recorded by high-speed scanners that associate the response associated with a filled-in circle with each question. Results are exported as ASCII flat files that are then stored in a database for further processing.

“Machine input” is another useful data collection means in the context of the present invention. Such input can be processed directly from automated machines used for laboratory and other functions (for example, hematology instruments and blood chemistry analyzers). In this case, an ASCII or similar file is often provided that can be imported directly into the system of the present invention for validation and further processing.

Tablet-based means also are suitable for data collection in the present invention, as mentioned above. Any form of electronic capture of a stylus that indicates a response can be stored as an ASCII file. The common feature of such tablet-based recording forms is a screen that presents a question to the user, and a stylus or keyboard by which responses can be recorded. Personal digital assistants (“PDAs”) are a typical example of such tablet-based means.

Extensible Markup Language (“XML”) data also could be used in conjunction with the data collection means of the present invention. Responses to questions would be tagged (with XML) in a manner that specifically indicates the question with which each response is associated. For example, the Clinical Data Interchange Standards Consortium (CDISC) provides a form of collecting data in a manner that utilizes a defined XML standard format.

It is understood that the above examples of data collection technologies and data transfer methods are meant to be representative, but non-limiting. In general, any form of data that can be converted into common data formats (e.g., comma separated values and the like) can be used in conjunction with the present invention.

### Centralized Database Functions

As discussed above, the present invention simplifies and improves on previous practices in field monitoring to assure that data quality and relevant clinical guidelines and requirements are met, such as Good Clinical Practices and recommendations of the International Committee on Harmonization. The invention accomplishes this by allowing much of what formerly had to be checked in the field to be checked, instead, at a central location, thus saving considerable travel time and travel expense, as well as proving the degree of quality monitoring and enabling standardized processes to be utilized throughout a study, development program, and enterprise.

The invention allows close tracking of progress and quality indicators at remote sites as well as the ability to rapidly and electronically assure data quality. These capabilities reduce the need to travel to sites to assess these performance measures, and thereby enable field monitoring resources to be allocated according to need rather than the current practice of regular visits. Even when these visits do occur, field monitors in the past have been hampered in their ability to effectively manage the sites for which they are responsible, because they lack performance indices and more detailed information to know where the weaknesses in site performance may be.

When a broad range of performance indicators can be tracked at a central location, as advantageously provided by the present invention, less work needs to be done in the field. Since field work is done manually for the most part, without the aid of sophisticated analysis tools, databases and other programs that require substantial computing power, the invention improves the quality and efficiency of field monitoring, since much of this now can be performed at a central location where the requisite computational tools are readily available. Moreover, in accordance with the invention, a steady stream of performance indicators, in the form of feedback from a central location, enables individuals who manage the individual site and/or the entire study to be able to rapidly determine individual sites' strengths and weaknesses, as well as study design problems that may be common to all participating sites. Finally, the invention's improved utilization of a central processing facility enables managers and monitors to better focus and allocate their resources according to the frequency and severity of problems that do occur. In particular, expensive field visits by highly trained individuals can be reduced, because many issues can be resolved before these develop into full-blown problems. Even when field visits do occur, managers and field monitors can go to each site with a clear idea of where their efforts need to be focused for optimal effectiveness.

The primary functions of the centralized database (i.e., the primary functions of the central processing site computers 8 described in connection with FIG. 1) according to the invention are input of data, digestion of data (including data validation and site management), and reporting of data.

Incoming data are batched and undergo a series of validation checks, including (1) range, (2) consistency with other answers (for example, if subject is male, then questions about pregnancies should have “blank” answers), and (3) trends or consistency across visits (for example, a sudden drop in hemoglobin level even though levels may be within normal ranges).

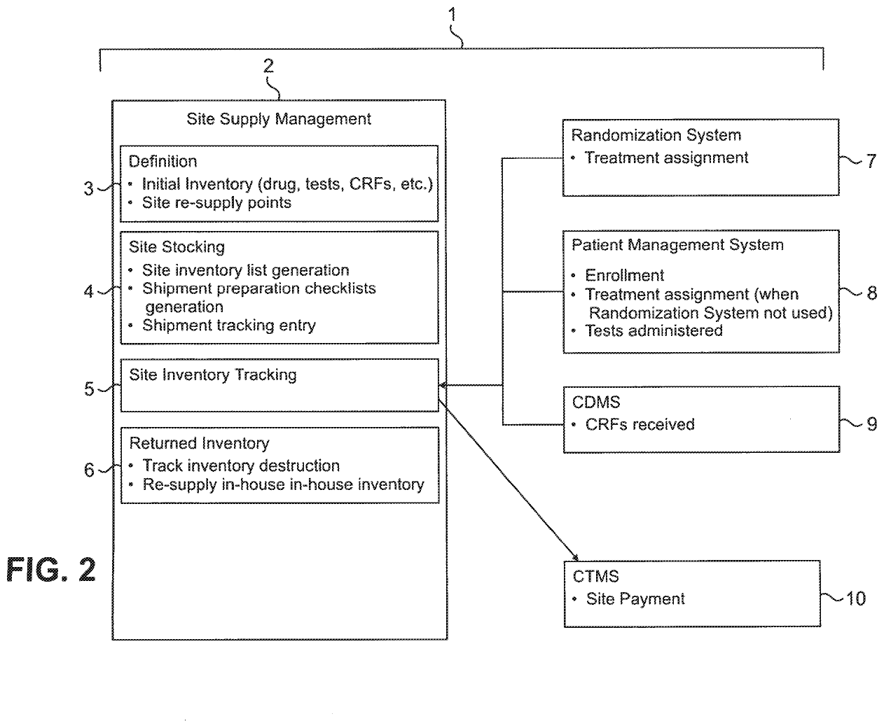

Various forms of site management pursuant to the present invention will be described in conjunction with FIGS. 2 through 5.

A system for site supply management is shown in FIG. 2. Sites must have adequate supplies with which to conduct a study, including Case Report Forms (CRFs), drugs, and other elements. Such supplies typically are sent out at the beginning of each study and may be either used up or discarded, as when errors are made that make forms or other supplies unusable.

As depicted in FIG. 2, site supply management system 1 comprises supply management computer system 2 which, in turn, contains a plurality of modules. Definition module 3 defines the initial inventory for each clinical site, and also defines the appropriate levels (“supply points”) at which the various supplies should be replenished. Site stocking module 4 is a database that maintains updated lists of the existing inventory of supplies at each clinical site, the supplies to be shipped to each site, and the supplies already shipped to each site. Site inventory tracking module 5 tracks supplies that are sent out to each site; automatically decrements these as patients are enrolled and progress through the study; and provides automated notification when replenishment is needed. Such notification typically is based on:
Rate of use. This can be determined based on data received from patient management system 8 (which is the same patient management system described in connection with FIG. 1). Patient management system 8 typically provides information about enrollment in the particular study, treatment assignment of patients, and the tests actually given to the patients.
Proximity to end of study. This can be determined based on data received from clinical data management system (“CDMS”) 9 (which is the same data collection means described in connection with FIG. 1). CDMS 9 indicates exactly how many CRFs have been received to date.
Randomization block size. This can be determined based on data received from randomization system 7 (which is the same randomization system described in connection with FIG. 1).
   Based on such notification, site inventory tracking module 5 provides the additional capability of triggering payments to the respective sites at appropriate times, via site payment system 10, which will be described further below. Finally, site supply management computer system 2 also contains returned inventory module 6, which tracks the destruction and/or return of supplies previously provided to clinical sites.

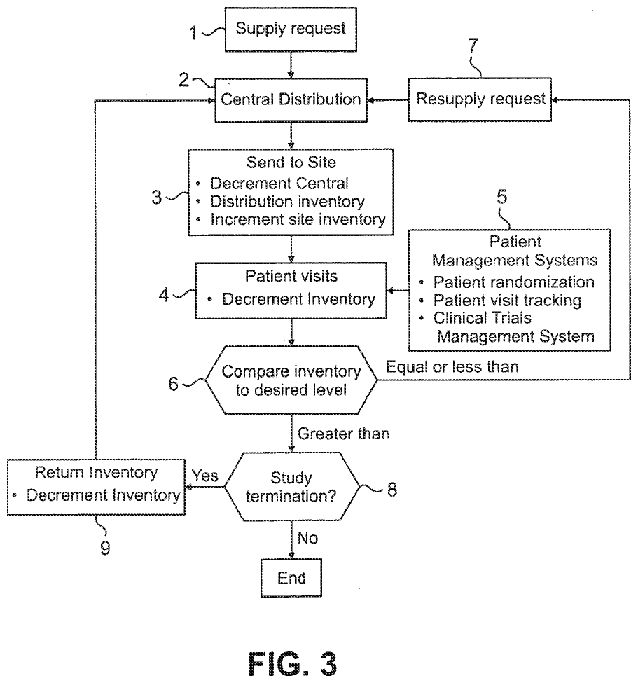

The operation of site inventory tracking module 5 of FIG. 2 is elucidated in the flow diagram of FRI 3. This portion of the invention's e g ent functionality uses information about patient visits and other elements to supply and periodically resupply study supplies that might include Case Report Forms, study drugs, laboratory kits, or other types of supplies. Each of these types of supplies can be tracked independently. The process, as shown in FIG. 3, begins with initial supplies sent out 1, normally at the time a site begins involvement with a study. These supplies are sent from one or more central distribution centers 2 such as the company managing the trial, drug packagers, or other suppliers. At the time study supplies are sent, the supply at both the distribution center and the study site is tracked 3, so central supplies are decremented and supplies on hand at the site are incremented. As the study progresses, different supplies may be consumed at different rates, and the appropriate site inventories are decremented. For example, at the time of patient screening for study suitability, laboratory kits and certain Case Report Forms may be used, but no drug distributed; at the time of enrollment into the study, however, Case Report Forms will be used and drug distributed, but additional laboratory kits may not be used. Each of the study supplies is inventoried and decremented 4 as particular study visits occur, with information about use coming from other components 5 described in the context of FIG. 2, such as randomization system land patient management system 8 shown in FIG. 2. As each component supply is used, a comparison 6 is made between supplies remaining and a predetermined desired level. This desired level can be adjusted during the course of the study, so that, for example, towards the end of a study, fewer supplies are kept on hand. Or, if the study drug is expensive or in limited supply, that element may be kept in reduced levels compared to initial supplies. These levels can also be established for each site, so that, for example, faster enrolling sites maintain a more generous supply of study materials than slower enrolling sites. At the point where study supplies reach the predetermined minimal level, an automatic resupply request 7 is sent to the central site. At the end of a study 8, when supplies are to be returned to a central site, a request to the site can be generated 9. Those supplies returned may be destroyed or, after quality assurance checks, redistributed. This option also provides for the unusual circumstance that study supplies may be redistributed during the course of a study, for example from a slowly enrolling site to other, faster-enrolling, sites. Under this scenario, supplies are similarly returned to a central site and quality assured before they can be redistributed. Manual corrections can be entered at the study inventory level. For example, if a study drug spilled, or Case Report Forms ruined, a manual system allows corrections to the site inventory. The system also allows tracking of resupply shipments. If problems occur, the system will generate emails to appropriate study personnel to ensure site supply problems are addressed in a timely fashion.

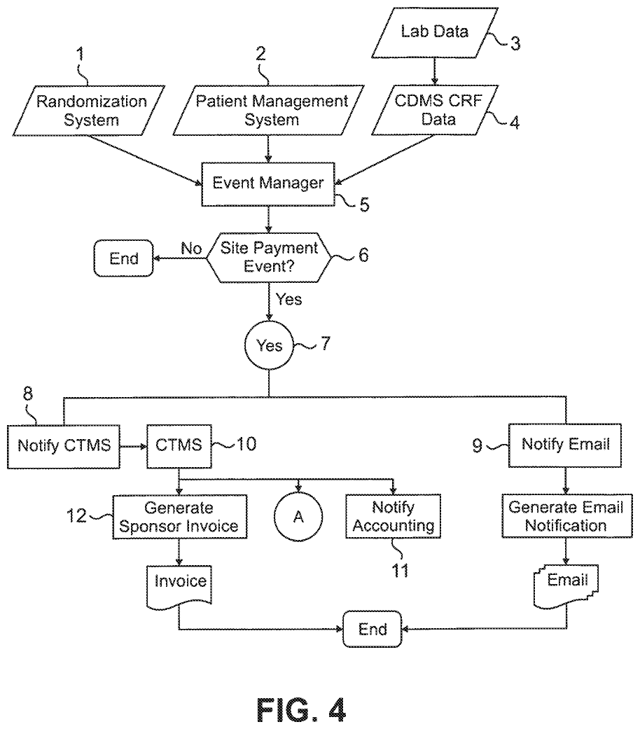

The operation of an embodiment of site payment system 10 of FIG. 2 is depicted in the block diagram of FIG. 4. Clinical sites typically are paid according to certain milestones, including individualized patient visits, in addition, some sites may perform optional services such as specific imaging (CT, MRI, or PET scans, for example). The invention provides the capability of making payments that are based on triggers established by certain events, such as visits, as well as when optional services are performed. In each case, the trigger for payment is based on receipt of data indicating that specific services, visits, or other measurable performance milestones have been met. Thus, in FIG. 4, the site payment system utilizes input from different components, including patient randomization system 1, patient management (i.e., visit tracking) system 2, laboratory data 3 and internal clinical data management system (CAMS) 4, in each case through a general service Event Manager 5 that monitors study data and triggers specific actions (“events”) when predefined study data parameters are achieved. Once an event is triggered 6, one or more predefined actions 7 can occur. Example actions include email notification, database updates, transaction updates to third party systems, and the like. A simple example of an event and triggered action is if a subject is enrolled in the clinical trial, the event manager will trigger an event 8 and the predefined action of an e-mail notification to study personnel will occur 9.

As is clear from FIG. 4, study sites are typically paid when site subject milestone events occur. An example of such a milestone would be when the tenth subject is enrolled at a clinical site 10. The Event Manager 5 is a flexible system, allowing milestone events to be based on any site data contained in the study database. Typically payments occur when a pre-defined number of subjects are screened, subjects are enrolled, or CRFs are received. Randomization system 1 provides Event Manager 5 with information about screened subjects and enrolled subjects for the clinical site. Patient management system 2 provides Event Manager 5 with detailed information about site subject visit activity, including subject visit dates, lab tests administered, and (when randomization system 1 is not used), screened and enrolled subjects. CDMS 4 provides Event Manager 5 with the number of CRFs received for a site and number of outstanding queries.

Once Event Manager 5 triggers a site payment event 7, any number of actions can occur. Typical trigger actions for site payment include a notification to the clinical trial management system (CTMS) 10 that a site payment needs to occur. CTMS systems are typically responsible for generating the payment (e.g., invoice or check) 12 and notifying the accounting system 11. As discussed above, the site payment event will also send an e-mail notification 9 to the study project manager and the CRA that a site payment is to occur and to generate a sponsor invoice as indicated 12.

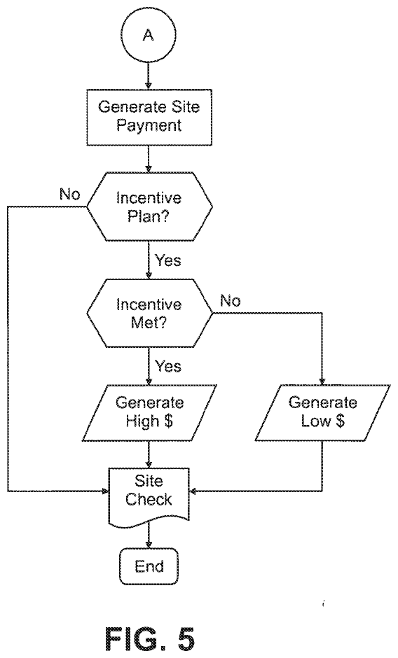

In addition, the invention provides the capability of implementing flexible financial incentive schemes to encourage sites to optimize their performance on measures such as queries (that is, data submitted that fail validation and therefore roust be returned to the site for clarification). One example, of such a financial incentive scheme is shown as a flow chart in FIG. 5, which generally shows the operation of an automated system for providing financial performance incentives to individual clinical sites. If the applicable financial incentive has been met in FIG. 5, the system generates a “high” payment to the particular clinical site. However, if the financial incentive has not been met in FIG. 5, the system generates a “low” payment to the clinical site.

In a specific embodiment of the automated system of FIG. 5, a complex set of payments is triggered such that a clinical site receives payment of, for example, 80% of fees for a given patient visit and for completion of data collected at that visit, and receives the 20% balance of the fees only when all the queries associated with that specific visit are met. Further examples of such incentive schemes might include paying sites (1) a bonus for having a low query rate, (2) incremental increases based on number of patients or rate of patient enrollment, and the like. The financial performance incentives of the invention can be further refined to include performance measures that are compared with other sites participating in the same (or another) study. For example, such an incentive system might enable the top 10% of performers e.g., those having the lowest query rates, the highest rates of patient enrollment or retention, etc.) to receive a bonus or reward, either monetary or otherwise, for their performance compared to other sites participating in the clinical trial. Sites in the top 20% might receive a different level of bonus, and so on. The benefit of this system according to the invention is that it is entirely automated, as illustrated in FIG. 5.

### Reporting Functions

Monitoring site performance with respect to various parameters is one of the most important aspects of study performance. Knowledge of a variety of performance metrics in real- or near-real time is crucial to optimizing study performance, because this knowledge enables continuous adjustments of many parameters. The several essential tools provided by the present invention are: (1) leans for timely collection of data and performance metrics, (2) means for summarizing data in different ways that are useful to different audiences, (3) means for intervening in response to issues that can be improved, and (4) means for monitoring the effect of any such intervention. These tools will be described in detail in the following paragraphs.

### (1) Timely Collection of Data and Performance Metrics

The present invention achieves the timely collection of data through flexible data collection methods, as described above. While the description above focuses on primary data, the invention also advantageously facilitates the tracking of meta-data (data about the data), which enables elements such as rate of patient re-enrollment, query rates, time to respond to queries, and many other performance indices to be tracked, on a site-by-site basis, in real time.

### (2) Summarizing Data

Data may be summarized, in accordance with the invention, in a manner that is meaningful to different audiences, such as site monitors, project managers, and program managers. For example, a site monitor may be most interested in the number of queries outstanding and how long each has existed, or in the number of different data fields that need source data verification (since this information reflects the site performance and affects when the next field monitoring trip needs to be scheduled). In contrast, a project manager may be most interested in tracking the number of, and reasons why, patients fail to meet screening criteria for entry into the study, as well as the number of, and reasons why, patients drop out of a study. On the other hand, a program manager may focus on overall enrollment and determining when study enrollment will be completed and when the study is projected to be completed. Since data are entered into a database, both data and performance metrics concerning the data can be measured and stored. These reports are web-enabled (e.g., utilizing web reports module 14 in FIG. 1) so that a variety of users (determined by functional role and permissions) can view such reports on-demand at any time, from any location at which internet access can be secured. This same information can be made available through SMS Text Messaging, RSS feeds, or any other form of wired or wireless connection (utilizing, for example, module 15 shown in FIG. 1).

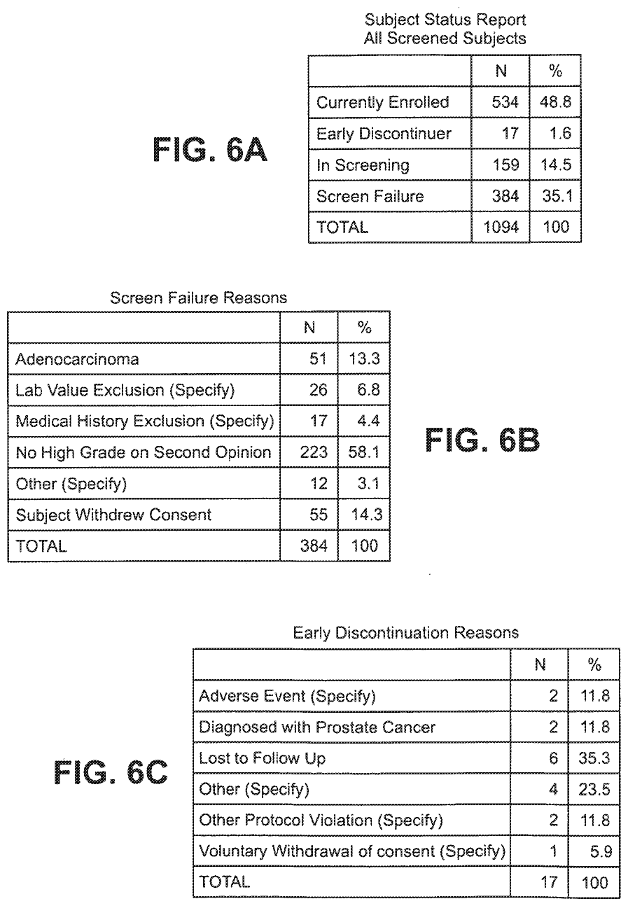

FIGS. 6A through 6C depict preferred reporting means, according to the invention, for monitoring certain study performance parameters that would be of interest to a project manager, as described in the preceding paragraph, Thus, FIG. 6A depicts a computer-generated table showing examples of performance measures that can be routinely tracked, such as the numbers of patients currently enrolled in the applicable study (“Currently Enrolled”), the number of patients who dropped out of the study prematurely (“Early Discontinuer”), the number of potential patients being screened for participation in the study (“In Screening”), and the number of potential patients who, for some reason, could not be enrolled in the study (“Screen Failure”). This overview information, as presented in FIG. 6A, can also be “drilled further down” into supporting details. For example, FIG. 6B is a computer-generated table showing the number of subjects who are not eligible for study participation because they fail to meet inclusion and/or exclusion criteria. These subjects, referred to as Screen Failures, may reflect limitations in the protocol, in procedures common to the entire study, or possibly a lack of understanding or other issues at the site level. In order to better understand why screen failures are occurring, a user can drill down by requesting a listing of reasons, as shown in FIG. 6B, why patients were not enrolled in the applicable study. Similarly, project managers might wish to know why patients drop ort of a study, and the reasons for doing so can easily be determined in accordance with the invention (see computer-generated table of “Early Discontinuation Reasons” depicted in FIG. 6C).

The reporting systems provided by the invention are highly flexible, so that standard reports can be modified quickly, or new ad hoc reports can be generated easily with minimally trained personnel. One of the challenges inherent in clinical research is that many complex and unforeseen circumstances occur, and this impairs the ability to manage such studies in direct proportion to the slowness in realizing and responding to such changing circumstances. Accordingly, the invention allows use of any standard relational database and employs normalized database design principles. The result is a database schema that is highly adaptable and is easily reported against, using standard database query tools such as Oracle or SAS or third party report writer tools, many of which are freely available in the market place, such as Crystal Reports, ReportsBuilder, and WebTools. Database tools, especially the more sophisticated report writing tools, make it possible for personnel with basic database training to create reports detailing complex study data relationships and study scenarios. Thus, if an unexpected laboratory abnormality occurred during the course of a trial, database tools could be used to prepare a new set of reports that can be posted to the study website through use of HTML or the like. This capability of quickly adding or modifying reports is a key advance over previous systems, where such adjustments could take weeks. For example, if standard study reporting appeared to indicate that a subpopulation of the study participants were responding negatively to a drug, an ad hoc report could be generated by frontline clinical personnel. Data could be generated based on demographic data elements such as gender, age, race or even region of the country. In accordance with the invention, the data would be compared, and if necessary, modifications to the study would be made. Additionally, if a standard report highlights a potential concern, but the data reported are incomplete, the reports can be easily modified using the same report writing tools in accordance with the invention, to include necessary, additional reporting criteria. A distinct advantage of the invention is that it permits such reporting changes to be made rapidly—often within minutes—and reports can be immediately deployed to the web and made readily available to the study team

### (3) Intervention Based on Performance-Related Data

The invention provides the additional, essential capability of intervening in response to issues that can be improved. Knowledge of a problem or issue (based on certain data collected and summarized as set forth in the preceding paragraphs) sounds an alarm to intervention, which can be implemented through any conventional manner appropriate to the circumstance and problem. For example, if it becomes apparent that a single question on a Case Report Form produces an inordinate number of queries or poor quality data, the wording of such question can be re-evaluated. If the clarity of the question can be improved, such question can be re-worded as appropriate. As another example, if the data somehow indicate that an individual site is having problems, the invention enables a site monitor or project manager to “drill down” in order to better understand the source of such problems. Thus, the site monitor or project manager may detect the data that one particular interviewer has a higher query rate than others at such site or elsewhere within the study. In that case, it is possible for the manager to intervene to determine whether the problem is one of inadequate training, time, or other factors with respect to such interviewer. As yet another example, if the data indicate that the enrollment rate is slow across all sites, a suitable intervention may include the addition of more sites or, if such data lead to the identification of elements at one or more sites amenable to further exhortation, training, threats, or other measures, the invention facilitates the determination of an appropriate intervention.

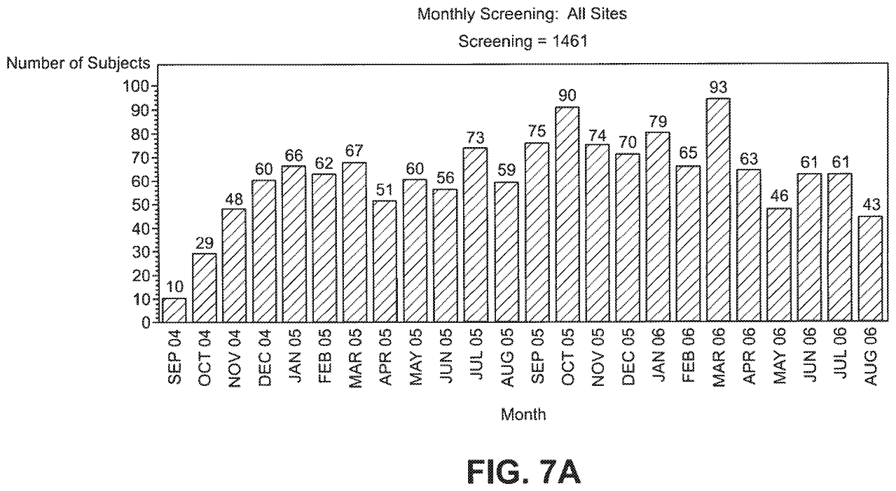

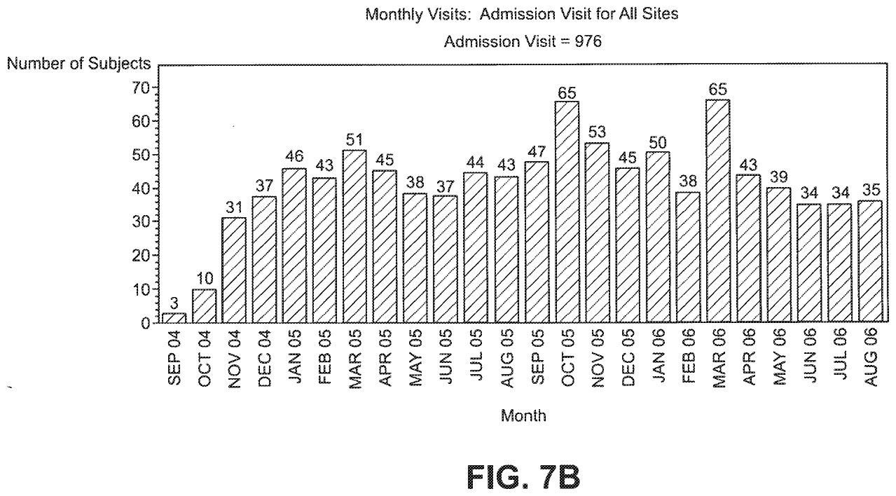

Examples of management intervention as a result of real-time performance tracking are demonstrated in FIGS. 7 and 8. Screening and enrollment over certain time periods can be readily tracked to identify temporal and other elements that may be amenable to improvements, such as the need to periodically reinvigorate clinical sites by call, letters, visits, or other means of managing a situation. The difference between screening and enrollment (i.e., the success rate of screening) can similarly be tracked to identify certain situations where such could be improved. Thus, the bar graphs in FIGS. 7A and 7B show, respectively, screening and enrollment visits in a particular study, by month. The former are visits where individuals are assessed for suitability to be included in the study, and the latter are those visits in which patients are actually enrolled. Comparison of these two metrics, including tracking the ratio of screened to enrolled patients, may reveal elements that are important for study management. For example, a high ratio overall may indicate an unreasonable screening criterion such as a laboratory test that might be relaxed or waived to enable more patients to be entered in the study. Individual sites can also be assessed, and those sites that are found to be screen-failing (i.e., failing to enroll) a disproportionately high number of patients relative to other sites can be further evaluated to determine the reasons for their poor performance. For example, it may be determined that the personnel at such sites have a poor understanding of what type of patients should be considered for inclusion in the study. Once this particular problem has been identified, the invention would facilitate an immediate solution: additional educational efforts. In this case, field monitors either would spend additional time on the telephone with such site and/or increase the urgency of a site visit.

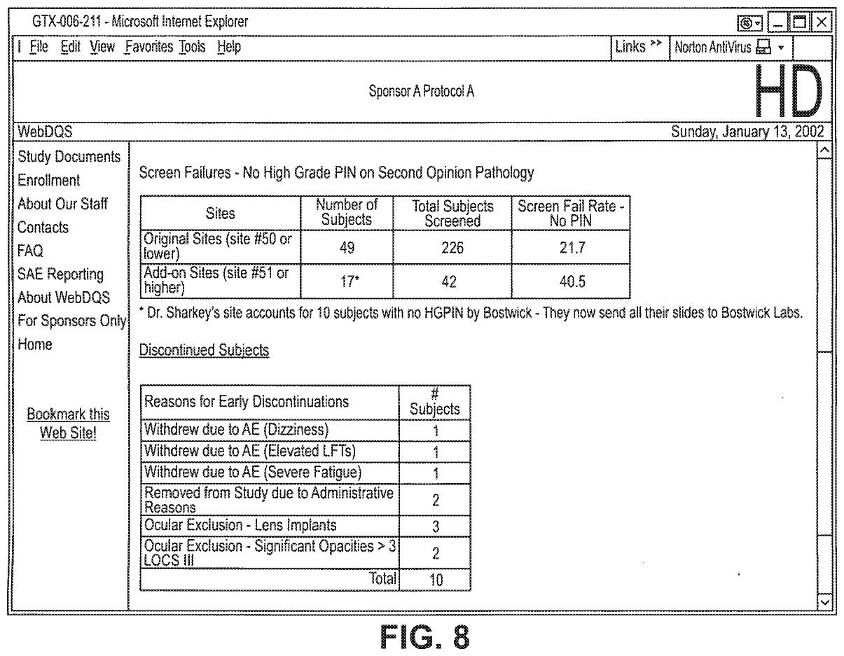

Another example of management intervention according to the invention is demonstrated in FIG. 8. In this case, the screen failure rate has been tracked over time and has been found to be progressively increasing during the study, indicating a progressively worsening problem with determining patients suitable for admission to the study. Moreover, the number of screening visits was unexpectedly high when compared with the resulting number of admission visits as the study progressed. Apparently a higher than acceptable number of individuals were being screened, because too large a proportion of these individuals did not qualify for study admission. Since this discrepancy was increasing over time, this alert would enable study managers to perform further investigation based on data communicated over the Internet, as exemplified in FIG. 8. The data shown in FIG. 8 indicate that newer sites, that were added after the study had started, had a screen failure rate substantially higher than that experienced by sites that had been in the study longer. Based on such data, managers could recognize this as a problem of insufficient training and be able to address the situation by immediate and forceful additional training, with the result that the screening failure rate could drop in those sites in which it had been a problem. The consequence would be that enrollment rates would improve and the study enrollment—one of the key limiting factors for any study and a major financial consideration—would be completed more quickly than would have been the case had the problem not been identified and intervention been taken. As further shown in FIG. 9, such information (e.g., the number of prospective patients screened) can also be provided on a site-by-site basis in accordance with the invention.

### (4) Monitoring the Effects of Intervention

Since a broad variety of measures may be tracked in accordance with the invention, as described above, the individual performance criteria that first brought attention to the problem can be tracked over time to measure improvement. Similarly, because the invention provides the capability of easily instituting further reports and indices of data and meta-data, as also described above, additional measures can be tracked as well.

### Site Interactions

An essential part of working with clinical sites is to assure that accurate information has been collected. When data are submitted from sites to the processing location database, these first undergo validation as described above. Errors and other discrepancies identified through this process are reviewed, and then released to sites via a web-enabled system via which sites can view queries, respond to them, and manage queries. (This is identical to the data query management module described in connection with FIG. 1.) The same capabilities of viewing and managing sites are also available to other study personnel, including the site managers, who can assist with query management and also gain insight into site operations by viewing the systems.

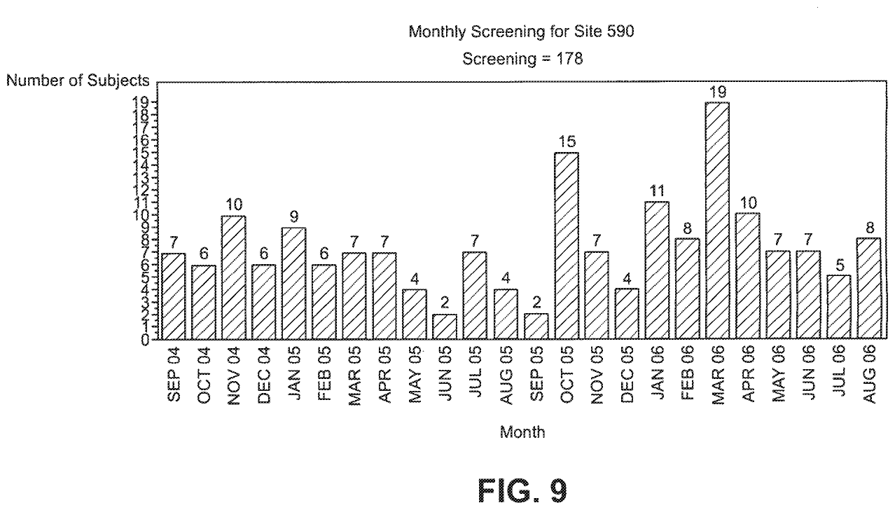

The means for query management and resolution, as contemplated by the invention, includes three major elements: The first element is the capability of viewing ail queries. After queries are released to sites, the sites are notified that new queries are awaiting or that their data generated no new queries. The site personnel then can log onto a website and see the list of queries awaiting resolution. An example of such a web interface is shown in FIG. 10, which depicts all new queries within the query management system. Users can also sort queries according to how long each has been outstanding, those that are overdue, and other means, utilizing, for example, the “query options” panel in the left gutter of FIG. 10. This system also enables users to print out a list of outstanding queries so that these can then be carried to different locations in an effort to determine missing or correct information.

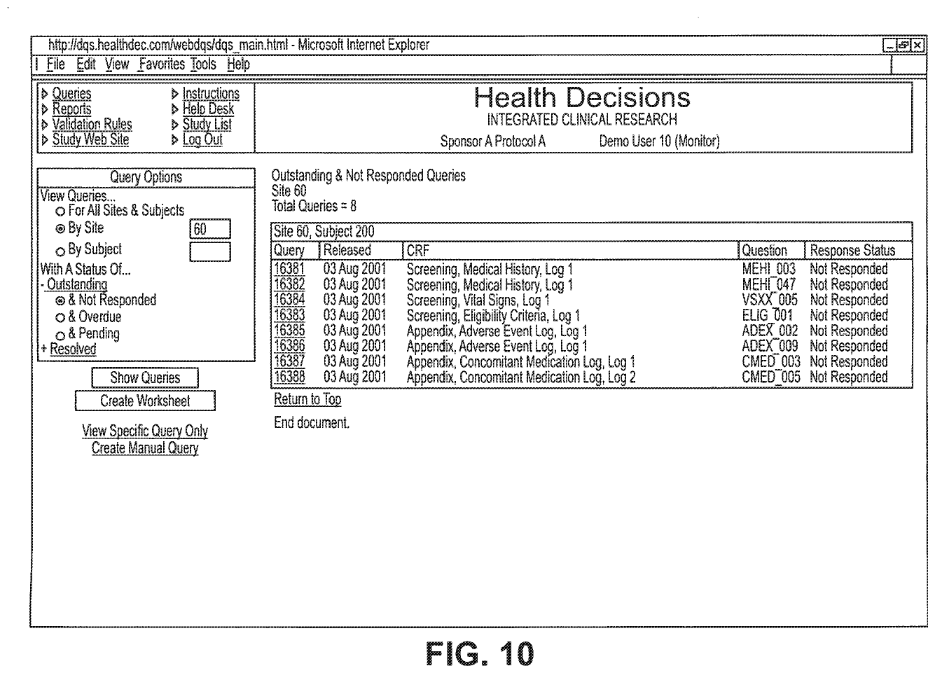

A second element of the query management system according to the invention is that queries can be responded to online, using a web interface such as that depicted in FIG. 11. When the user has information to be entered, the user can bring up an individual query and indicate a response, including the possibility that information may be unobtainable (so that the query is irreconcilable).

The third and final element of the inventive query management system is the ability to write manual queries. Site monitors may use this system for writing manual queries while at the site or any other another location. Each change is accompanied by a full audit trail (including such information as previous value, new value, who is changing, date/time, reason for change, etc.).

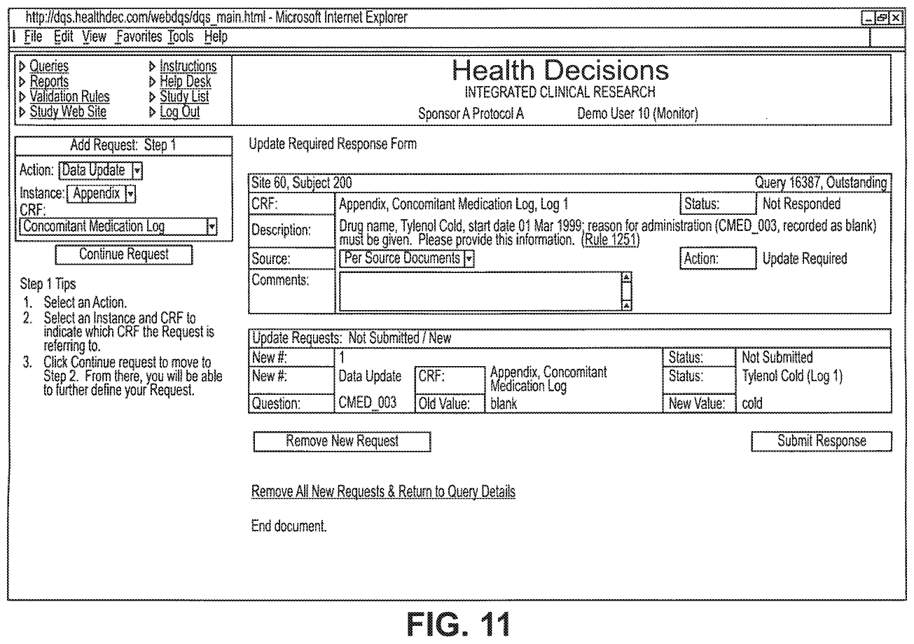

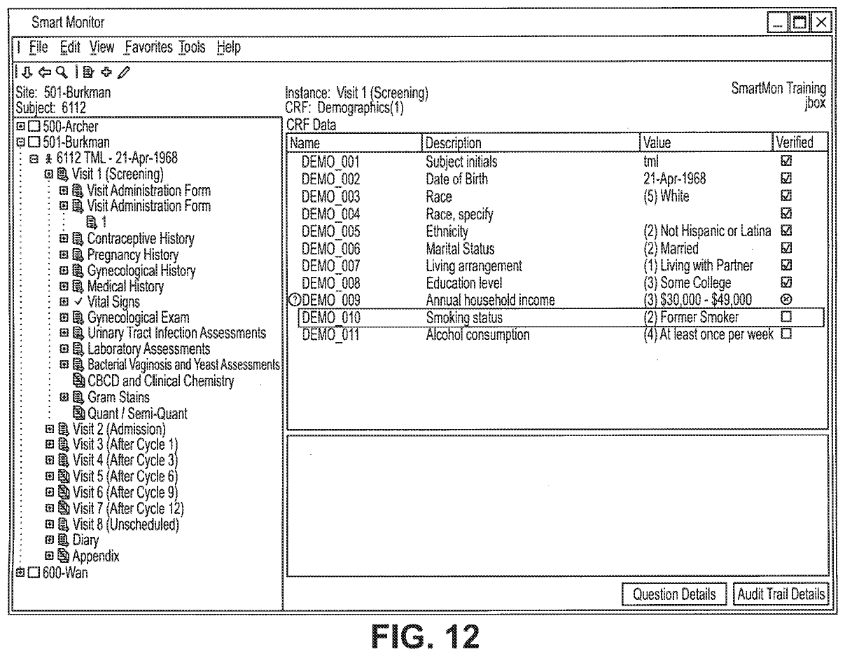

As noted previously, one of the inherently challenging aspects of clinical research is that the complexity of evaluations and, often, the geographic dispersion of many different sites, means that unanticipated and complex issues may arise. While, on an individual basis, some of these may be relatively minor (for example, a delay of several weeks before inputting into the system data that have been collected from patients), these issues are important collectively, in that it becomes quite difficult to manage complex systems without prompt knowledge of exactly what is occurring at these sites. This concept is illustrated in FIGS. 12-14, which show several representative performance metrics by which site and study performance can be tracked. For example, FIG. 12 depicts a computer-implemented embodiment of the invention, indicating exactly which data fields have been monitored at a particular site (in this case, site number 501) and which fields continue to require monitoring at such site. (The particular data fields highlighted in FIG. 12 relate to demographic data.) In accordance with the invention, data of the type shown in FIG. 12 may be aggregated and compared in the manner shown, by way of example, in FIG. 13, FIG. 13 provides a report directly comparing all sites participating in a study, with respect to (a) the total number of patients for whom data have been collected at each site, (b) the total number of data fields applicable to such patients, (c) the number of such fields for which the data have been verified, (d) the number of such fields for which the data have not been verified, and (e) a computation of the percentage of data fields that have been verified. In the aggregate (i.e., across all sites), according to FIG. 13, 79.5% of the half-million fields have been verified.

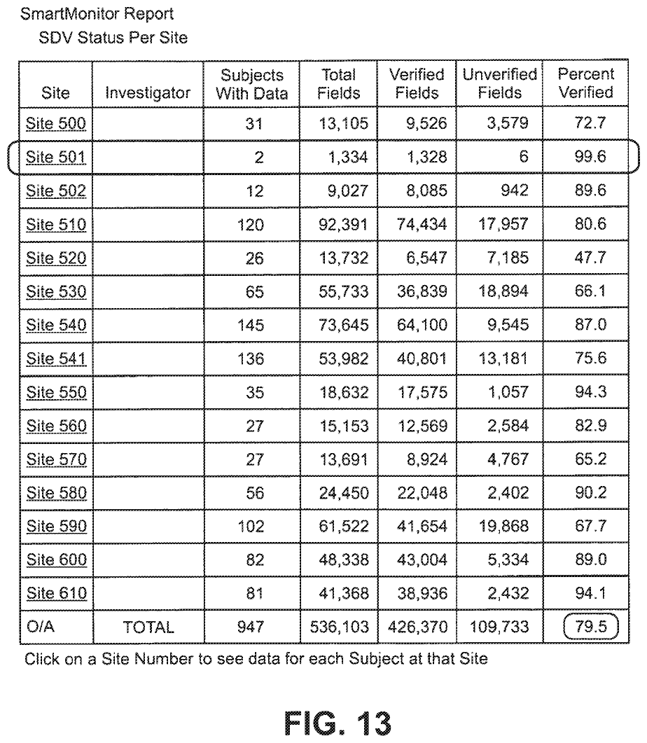

The invention provides the capability of further “drilling down” into the type of information presented in FIG. 13. For example, FIG. 14 provides detailed information for the two specific patients (“subjects”) enrolled at site number 501. As shown in FIG. 14, both of the patients (identified as subjects 6111 and 6112, respectively) have completed their participation in the clinical study. Complete data have been received and verified for subject 6112, as indicated by the “100.0” entry in the “Percent Verified” column. However, the data in certain fields remain unverified for subject 6111, as indicated by the “6” entry in the “Unverified Fields” column and the “96.5” entry in the “Percent Verified” column. Based on such data, a site monitor or other manager utilizing the system of the invention can readily determine whether/when a monitoring visit should be made to the applicable site. In addition, such data enable the site monitor to identify issues that will require attention during the next monitoring visit. Such knowledge thus enables intervention to manage the study, with different actions being indicated depending on the specific problems or weak areas reflected in the applicable performance measures.

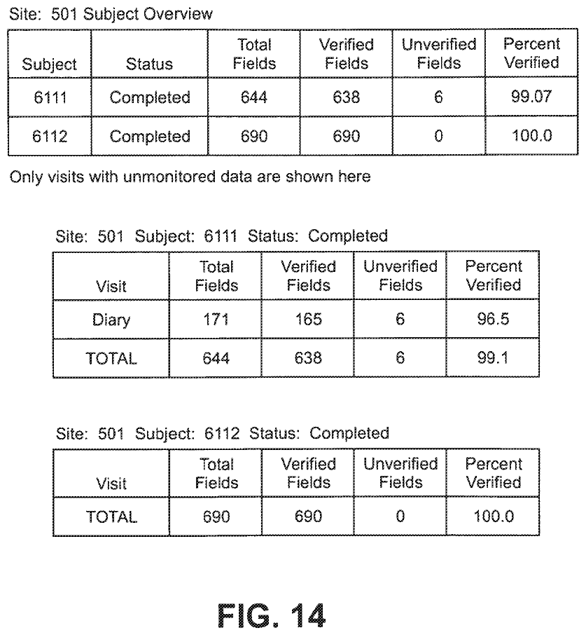

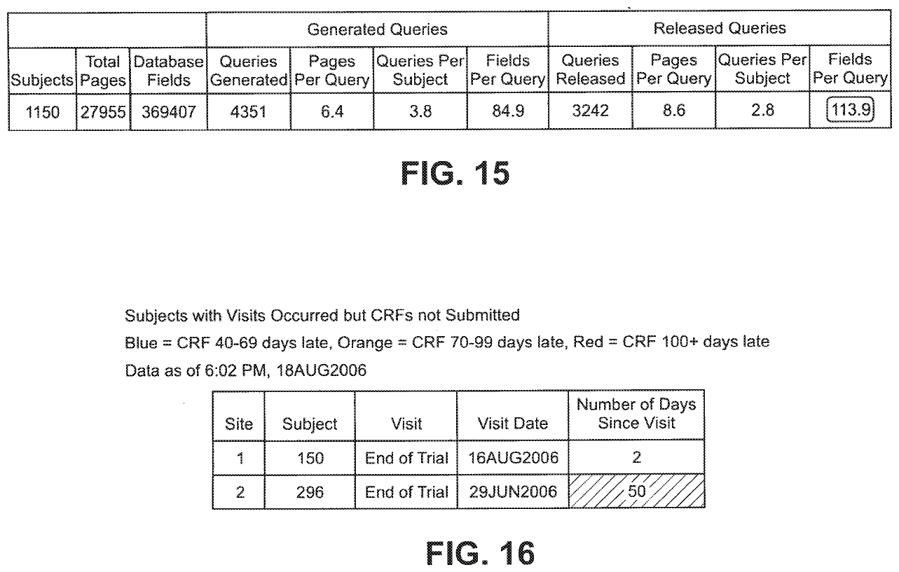

In a preferred embodiment, the invention enables a “Site Performance Index” as an overall measure of the respective sites' abilities to measure quality of data. This Index may include various measures such as query rate, time to respond to queries, and other measures that can together form a simple, convenient measure of how well the various sites perform, and thus guide oversight efforts. For each site, this information is compared against average values for all sites, and each field monitor can continuously track this as a key performance indicator. Two specific implementations of site performance measurement according to the invention are shown in FIGS. 15 and 16, respectively. FIG. 15 illustrates a reporting means, according to the invention, for tracking the numbers of data queries generated and resolved on a study-wide basis. A metric employed in FIG. 15 is that of “released queries,” which refers to data that could not be determined from the Case Report Form page submitted, so that queries must be released to sites for further and verification of such data. FIG. 16, on the other hand, illustrates a reporting means for tracking the timeliness of data submission with respect to patients whose enrollment in a study has ended. No CRF has been submitted with respect to the final (“end of trial”) visit by either of the two patients identified in FIG. 17. In the case of one of the patients (subject 150), the lack of a submitted CRF is not problematic, since only two days have elapsed since this patient's final visit. However, in the case of the other patient (subject 296), 50 days have elapsed since the patient's final visit. The fact that no CRF has yet been received, after such a lengthy time period, is “red-flagged” by the invention for follow-up by the appropriate site monitor or manager.

By minimizing the number of outstanding queries, the invention advantageously facilitates rapid “database lock.” Locking the database represents the culmination of efforts spanning months or years in a particular clinical study, and often represents the future of a product or even a company. This event requires that every query, and all outstanding discrepancies regarding data, have been resolved, “Database lock” times in the industry currently range between about several weeks and several months after the last patient last visit (“LPLV”), with an average of probably about eight weeks after LPLV. However, with careful planning, the integrated system of the invention potentially enables database lock as early as the same day as LPLV, with an average of about five to seven days after LPLV.

The foregoing description details certain embodiments of the invention. It will be appreciated, however, that the invention can be practiced in many ways. It also should be noted that the use of particular terminology when describing certain features or aspects of the invention should not be taken to imply that the terminology is being re-defined herein to be restricted to including any specific characteristics of the features or aspects of the invention with which that terminology is associated. The scope of the invention should therefore be construed in accordance with the appended claims and any equivalents thereof.

1-36. (canceled)

37. A computer-implemented method for conducting an adaptive clinical trial at a plurality of geographically remote clinical sites according to a set of procedures or parameters, said method comprising the steps of:
(a) collecting performance metric data with respect to the performance of a particular clinical site, performance of clinical personnel, or achievement of study parameters in the course of conducting said clinical trial at said clinical site;
(b) electronically reporting the data to a pre-programmed computer module;
(c) determining in real time, by use of said pre-programmed computer module, whether procedures or parameters utilized in conducting said clinical trial require modification;
(d) providing instructions, based on said determining, to modify the procedures or parameters utilized in conducting said clinical trial; and
(e) modifying the procedures or parameters utilized in conducting said clinical trial, based on said instructions.

38. The method of claim 37 wherein the performance metric data is a measurement of: error rates, time required to respond to errors or to data queries returned to a site, number of patients screened, number of patients enrolled, or length of time required to transmit data following a patient visit.

39. (canceled)

40. The method of claim 37 wherein said instructions in step (d) are to: make a partial or complete payment to a remote site, adjust the inventory of supplies for a remote site, change the schedule for re-supply of a remote site, or distribute additional supplies to a remote site.

41. The method of claim 40 wherein the data correspond to the performance of a remote site, and wherein the amount of payment to such site is determined based on said performance.

42. The method of claim 41 wherein the performance data comprise a determination of: rapidity of submission of data following a patient visit, accuracy of data, speed of resolution of data discrepancies or queries, or completion of specific medical or laboratory procedures.

43. The method of claim 37 wherein access and permissions to said reported data are controlled.

44. The method of claim 37, wherein said procedures or parameters comprise randomizing patient drug dosages or randomly assigning patients into treatment groups.

45. The method of claim 44, wherein said instructions in step (d) are to: assign patients to different treatment groups, suspend enrollment in one or more treatment groups, or change the scheduling of a field monitoring visit to a remote site.

46. The method of claim 37 wherein steps (b), (c) and (d) further comprise:
(i) transmitting, through the web, a description of each discrepancy or query relating to the data, the validation that triggered the query, and means for responding to such query;
(ii) measuring the number of queries per clinical site or per each question in a questionnaire; or
(iii) measuring response time of a clinical site to one or more queries.

47. A computerized system for conducting an adaptive clinical trial at a plurality of geographically remote clinical sites according to a set of procedures or parameters, said system comprising:
(a) means for collecting performance metric data with respect to the performance of a particular clinical site, performance of clinical personnel, or achievement of study parameters in the course of conducting said clinical trial at said clinical site;
(b) means for electronically reporting the data to a pre-programmed computer module capable of determining, in real time, whether procedures or parameters utilized in conducting said clinical trial require modification;
(c) means for providing instructions, based on said determining, to modify the procedures or parameters utilized in conducting said clinical trial; and
(d) means for modifying the procedures or parameters utilized in conducting said clinical trial, based on said instructions.

48. The system of claim 47 wherein the performance metric data is a measurement of: error rates, time required to respond to errors or to data queries returned to a site, number of patients screened, number of patients enrolled, or length of time required to transmit data following a patient visit.

49. (canceled)

50. The system of claim 47 wherein said instructions in paragraph (c) are to: make a partial or complete payment to a remote site, adjust the inventory of supplies for a remote site, change the schedule for re-supply of a remote site, or distribute additional supplies to a remote site.

51. The system of claim 50 wherein the data correspond to the performance of a remote site, and wherein the amount of payment to such site is determined based on said performance.

52. The system of claim 51 wherein the performance data comprise a determination of: rapidity of submission of data following a patient visit, accuracy of data, speed of resolution of data discrepancies or queries, or completion of specific medical or laboratory procedures.

53. The system of claim 47 wherein access and permissions to said reported data are controlled.

54. The system of claim 47, wherein said system further comprises means for randomizing patient drug dosages or for randomly assigning patients into treatment groups.

55. The system of claim 54, wherein said instructions in paragraph (c) are to: assign patients to different treatment groups, suspend enrollment in one or more treatment groups, or change the scheduling of a field monitoring visit to a remote site.

56. The system of claim 47 wherein the means in paragraphs (b) and (c) further comprise means for:
(i) transmitting, through the web, a description of each discrepancy or query relating to the data, the validation that triggered the query, and means for responding to such query;
(ii) measuring the number of queries per clinical site or per each question in a questionnaire; or
(iii) measuring response time of a clinical site to one or more queries.
